(19) United States
(12) Patent Application Publication
(10) Pub. No.: US 2007/0100404 A1
(43) Pub. Date: May 3, 2007

(54) Pad for cooling or heating

(76) Inventors: Hao-Chih Ko, Taipei County (TW); Chen-Heng Lin, Taipei (TW)

Correspondence Address: BACON & THOMAS, PLLC, 625 SLATERS LANE, FOURTH FLOOR, ALEXANDRIA, VA, 22314, US

(21) Appl. No.: 11/262,794

(22) Filed: Nov. 1, 2005

(51) Int. Cl. A61F 7/00

(52) U.S. Cl. 607/104

(57) ABSTRACT

The present invention is to provide a pad with a predetermined temperature, which has a plurality of elongate, curve channels formed on a top surface of the pad, the channels being connected together, wherein one end of the channel is formed as an inlet, the other end of the channel is formed as an outlet, the inlet and the outlet are provided at the same side of the pad, the channel has an arc-shaped periphery, a flat bottom formed on the top surface of the pad, and a lengthwise rib is formed along an inner surface of the channel facing the flat bottom; and a covering member provided on the top surface of the pad with the channels formed thereon, such that a cooling or heating fluid (e.g., water) may flow into or flow out of the channels via the inlet or outlet to keep the top surface of the pad maintaining at an optimum temperature.

### FIELD OF THE INVENTION

The present invention relates to pads and more particularly to such a pad adapted to be in fluid communication with a cooling device or a heating device so as to comfortably cool or heat the body of a person with the pad applied thereon.

### BACKGROUND OF THE INVENTION

Taiwanese Utility Model Patent Application, filed Nov. 13, 2003, entitled “Pad Structure For Cooling Or Heating”, now Taiwanese Utility Model Patent No. M243,119 discloses the following characteristics. A pad includes a plurality of curve channels connected together. A first coupling and a second coupling are provided at one side of the pad and they are connected to the channels. A third coupling is provided at the other side of the pad and it is connected to the channel. Also, a second channel is provided across top of the pad. The second channel has one end connected to the first coupling. A fourth coupling is connected to the other end of the second channel. In use of the pad, a user may insert plugs in the first coupling and the fourth coupling for sealing respectively. Next, connect inlet pipe or outlet pipe of a cooling device or a heating device to the second coupling and the third coupling respectively. Thus, fluid (e.g., water) may flow from the cooling device or the heating device to the channels via the second coupling. After cooling or warming the body, fluid may flow back to the cooling device or the heating device via the third coupling. Again, fluid is cooled or heated by the cooling device or the heating device prior to flowing to the pad. Surfaces of the pad thus may maintain at an optimum temperature by continuously performing the above cycle. As an end, a user may not have the feeling of too hot or too cold when using the pad. Advantageously, a product having the above characteristics of pad structure for cooling or heating have had market acceptance.

The present inventor is also aware that the only way for a manufacturer to survive in the competitive market is to produce products with novel and/or improved features by continuously dedicating to development. With respect to the above pad, it is comprised of an upper member and a lower member glued to the upper member. Also, each channel has a section of U formed integrally with the lower member.

However, the U-shaped channels are not easy to bend. Also, the channels may deform in response to applied pressure. As such, fluid may not flow smoothly. And in turn, surface temperature of the pad may not maintain at its predetermined value. This may greatly lower the desired cooling or heating performance of the pad. Thus, it is desirable to provide a pad for cooling or heating with improved channels in order to overcome the inadequacies of the prior art, increase market acceptance of the improved pad for cooling or heating, and provide a high quality pad for cooling or heating to the vast consumers.

### SUMMARY OF THE INVENTION

After considerable research and experimentation, a pad for cooling or heating according to the present invention has been devised so as to overcome the above drawbacks of the prior channel structure of the pad for cooling or heating discussed in the above background section and thereby meeting an increasing demand to the features and quality of the pad by the market and people.

It is an object of the present invention to provide a pad adapted to apply to the body of a person and thus the body temperature can be maintained at a predetermined optimum value for providing a degree of comfort to the body. The pad with improved channels provided on its top surface has the following advantages. Bending of the pad surface is easier. The channels may not deform in response to applied pressure. Further, fluid may flow smoothly in the channels. The characteristics of the present invention are that the pad has a top surface provided with a plurality of elongate, curve channels connected together. One end of the channel is formed as an inlet and the other end thereof is formed as an outlet. Both the inlet and the outlet are provided at the same side of the pad. The channel has an arc-shaped periphery and a flat bottom formed on the top surface of the pad. A lengthwise rib is formed along an inner surface of the channel facing the flat bottom. A covering member is provided on the top surface of the pad with the channels formed thereon for providing a flat top to the pad. An inlet coupling and an outlet coupling are provided at the inlet and the outlet of the channel respectively such that a cooling or heating fluid (e.g., water) may flow into the channels via the inlet coupling at the inlet. Next, the cooling or heating fluid flows out of the channels via the outlet coupling at the outlet after traveling the channels. Finally, the cooling or heating fluid returns to a cooling device or a heating device for cooling or heating again. Thereafter, the cooling or heating fluid may flow to the channels again. By utilizing this pad for cooling or heating, the top surface of the pad thus may maintain at an optimum temperature by continuously performing the above cycle. As an end, a user may not have the feeling of too hot or too cold when using the pad.

It is another object of the present invention to connect one pad having the above construction to the other identical pad side by side. A T inlet coupling is fitted in the inlet of one pad and the inlet of the other pad and a T outlet coupling is fitted in the outlet of one pad and the outlet of the other pad respectively. As a result, use area is increased and thereby maintain the top surfaces of the pads at an optimum temperature.

The above and other objects, features and advantages of the present invention will become apparent from the following detailed description taken with the accompanying drawings.

### BRIEF DESCRIPTION OF THE DRAWINGS

FIG. 1 is an exploded perspective view of a portion of a preferred embodiment of pad for cooling or heating according to the invention;

FIG. 2 is a top plan view of the pad;

FIG. 3 is a top plan view of two pads coupled together side by side; and

FIG. 4 is an exploded perspective view of a portion of the FIG. 3.

### DETAILED DESCRIPTION OF THE PREFERRED EMBODIMENTS

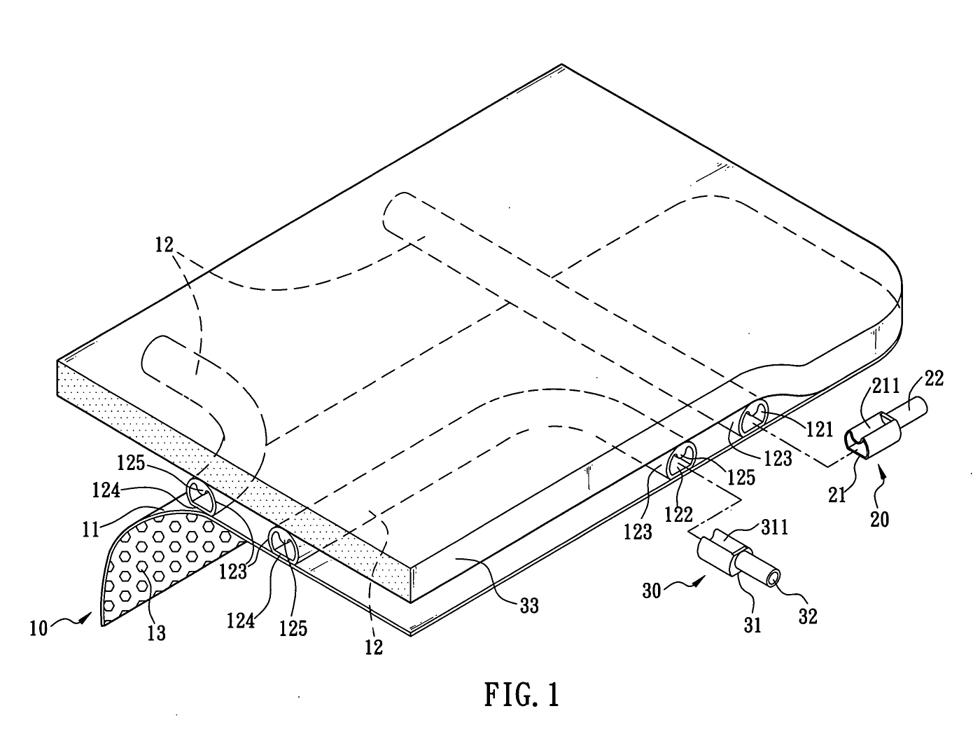

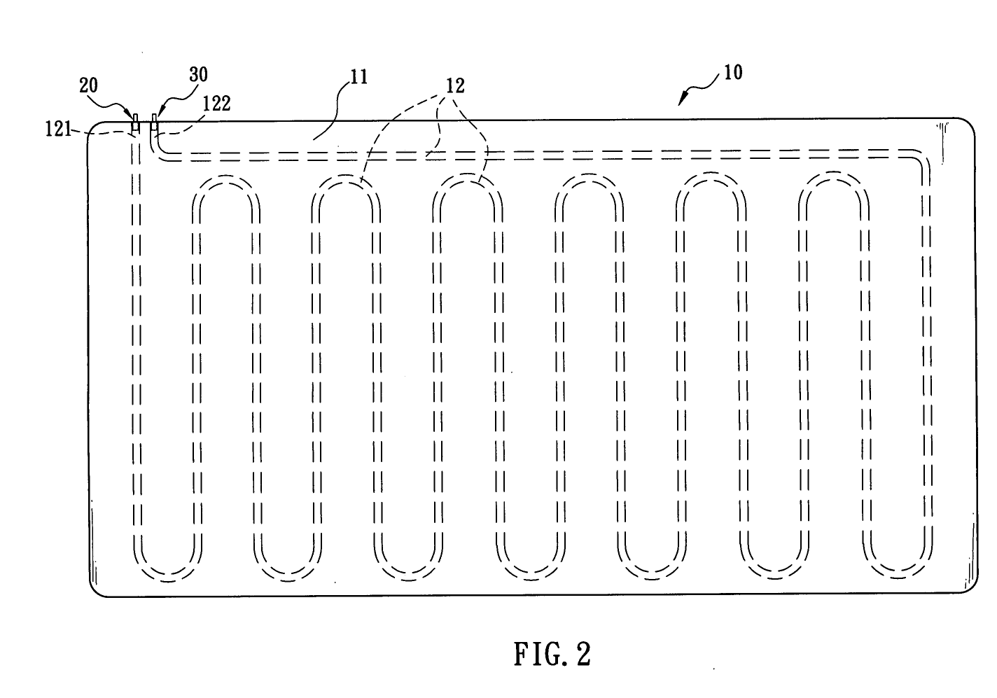

Referring to FIGS. 1 and 2, a structure for cooling or heating in accordance with a preferred embodiment of the invention is shown. The structure is adapted to apply to the body of a person and thus maintains the body temperature at a predetermined optimum value for providing a degree of comfort to the body. The structure comprises a pad 10 having a top surface 11 provided with a plurality of elongate, curve channels 12 of U-section connected together (see FIG. 2). One end of the channel 12 is formed as an inlet 121 and the other end thereof is formed as an outlet 122. Both the inlet 121 and the outlet 122 are provided at the same side of the pad 10. The channel 12 has an arc-shaped periphery 123 and a flat bottom 124 formed on the top surface 11 of the pad 10 (see FIG. 1).

Moreover, a lengthwise rib 125 is formed along an inner surface of the channel 12 facing the flat bottom 124. The arc-shaped periphery 123 is adapted to facilitate bending the channel 12 on the top surface 11 of the pad 10. Also, the flat bottom 124 is adapted to increase a contact area of the channel 12 and the top surface 11 of the pad 10. Further, the rib 15 on the inner surface of the channel 12 is adapted to increase a structural strength of the channels 12 so as to resist pressure applied on the channels 12. As an end, the channels 12 are prevented from being deformed. Otherwise, fluid flow in the channels 12 may be hindered or even blocked.

Referring to FIGS. 1 and 2 again, an inlet coupling 20 and an outlet coupling 30 are provided at the inlet 121 and the outlet 122 of the channel 12 in the pad 10 respectively. The inlet coupling 20 is identical to the outlet coupling 30 (see FIG. 1). Outer diameter of one end of the inlet coupling 20 is smaller than an inner diameter of each of the inlet 121 and the outlet 122. A flat bottom 21 and an upper groove 211 are formed at periphery of one end of the inlet coupling 20 and a flat bottom 31 and an upper groove 311 are formed at periphery of one end of the outlet coupling 30 respectively. The flat bottom 21 and the upper groove 211 are dimensioned and shaped to snugly insert into the flat bottom 124 and the rib 125 respectively for connecting the inlet coupling 20 and the channel 12 together. Also, the flat bottom 31 and the upper groove 311 are dimensioned and shaped to snugly insert into the flat bottom 124 and the rib 125 respectively for connecting the outlet coupling 30 and the channel 12 together. Further, the other end of the inlet coupling 20 is formed as a hollow, cylindrical section 22 and the other end of the outlet coupling 30 is formed as a hollow, cylindrical section 32 respectively for permitting cooling or heating fluid (e.g., water) to flow therethrough.

In addition, a covering member (e.g., sponge) 33 is formed on the top surface 11 of the pad 10 with the channels 12 formed thereon. The covering member 33 is adapted to not only cover the channels 12 for providing a flat top thereto but also provide softness and flexibility to the pad 10.

Additionally, a pattern (not shown) is printed (or painted) on a bottom surface 13 of the pad 10 opposite the channels 12. The pattern is adapted to increase aesthesia to the pad's external appearance so as to attract more people to buy it.

In view of the above configuration it is clear that the cooling or heating fluid may flow into the channels 12 via the inlet coupling 20 at the inlet 121. Next, the cooling or heating fluid flows out of the channels 12 via the outlet coupling 30 at the outlet 122 after traveling the channels 12 on the top surface 11 of the pad 10. Finally, the cooling or heating fluid returns to a cooling device or a heating device (not shown) for cooling or heating again. Thereafter, the cooling or heating fluid may flow to the channels 12 in the pad 10 again. Top surface 11 of the pad 10 thus may maintain at an optimum temperature by continuously performing the above cycle. As an end, a user may not have the feeling of too hot or too cold when using the pad 10.

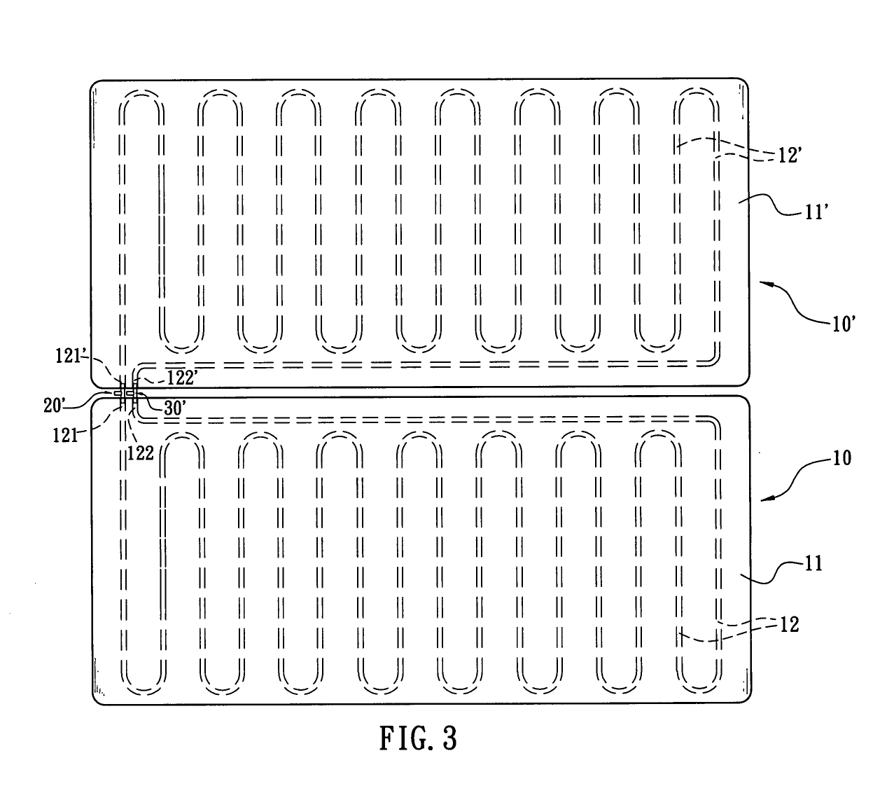

Referring to FIG. 3, a practical arrangement of the invention is illustrated. One pad 10 of FIGS. 1 and 2 and the other pad 10′ are connected together side by side. The pad 10′ is identical to the pad 10. The pad 10′ has a top surface 11′ provided with a plurality of elongate, curve channels 12′ of U-section connected together. One end of the channel 12′ is formed as an inlet 121′ and the other end thereof is formed as an outlet 122′. Both the inlet 121′ and the outlet 122′ are provided at the same side of the pad 10′.

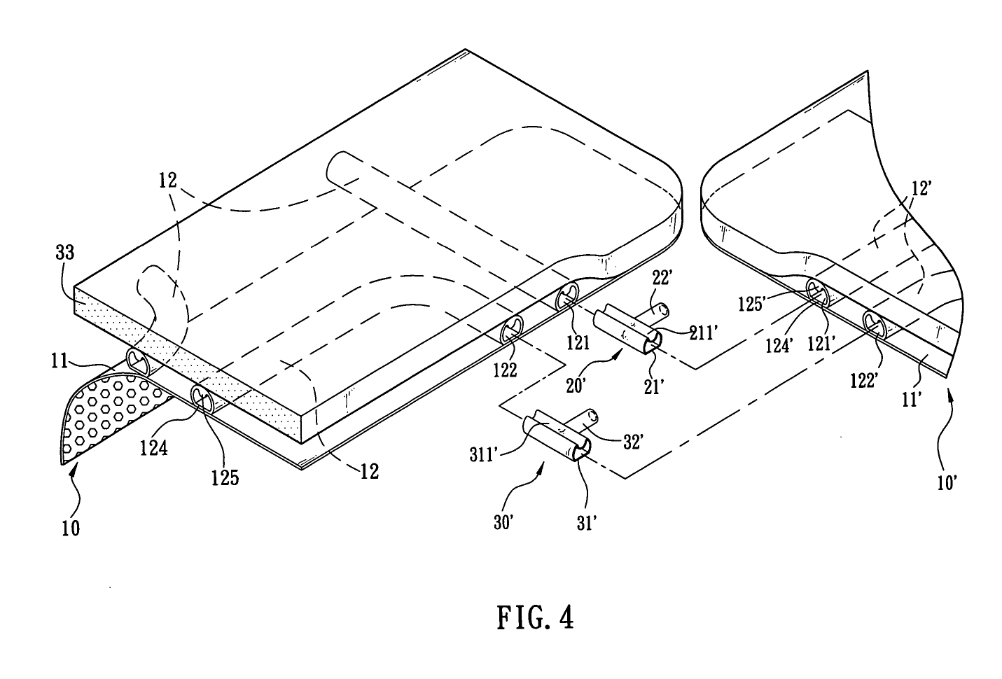

Referring to FIGS. 3 and 4, a T inlet coupling 20′ is fitted in the inlet 121 of the pad 10 and the inlet 121′ of the pad 10′ and a T outlet coupling 30′ is fitted in the outlet 122 of the pad 10 and the outlet 122′ of the pad 10′ respectively. As a result, the pad 10 and the pad 10′ are connected together. The T inlet coupling 20′ is identical to the T outlet coupling 30′. Outer diameter of either one of first and second ends of the T inlet coupling 20′ is smaller than an inner diameter of each of the inlet 121′ and the outlet 122′. A flat bottom 21′ and an upper groove 211′ are formed at periphery of either one of first and second ends of the T inlet coupling 20′ and a flat bottom 31 and an upper groove 311 are formed at periphery of either one of first and second ends of the T outlet coupling 30′ respectively. The flat bottom 21′ and the upper groove 211′ are dimensioned and shaped to snugly insert into the flat bottom 124′ and the rib 125′ respectively for connecting the T inlet coupling 20′ and the channel 12′ together. Also, the flat bottom 31′ and the upper groove 311′ are dimensioned and shaped to snugly insert into the flat bottom 124′ and the rib 125′ respectively for connecting the T outlet coupling 30′ and the channel 12′ together. Further, the third end of the T inlet coupling 20′ is formed as a hollow, cylindrical section 22′ and the third end of the T outlet coupling 30′ is formed as a hollow, cylindrical section 32′ respectively.

By configuring as above, the cooling or heating fluid may flow into the pad 12 of the pad 10 and the pad 12′ of the pad 10′ via the first and second ends of the T inlet coupling 20′ respectively after entering the hollow, cylindrical section 22′ thereof. Next, one branch of the cooling or heating fluid flows out of the channels 12 via the T outlet coupling 30′ at the outlet 122 after traveling the channels 12 and the other branch of the cooling or heating fluid flows out of the channels 12′ via the T outlet coupling 30′ at the outlet 122′ after traveling the channels 12′. Finally, the cooling or heating fluid returns to the cooling device or the heating device for cooling or heating again via the hollow, cylindrical section 32′. Thereafter, in another cycle the cooling or heating fluid may flow to the channels 12 in the pad 10 and the channels 12′ in the pad 10′ again. The cycle may continue for cooling or heating the pad as desired.

In view of above, a simple construction incorporating a pad 10 having channels 12 and another pad 10′ having channels 12′ as contemplated by the invention is embodied. The invention thus has the following advantages. Bending of the pad 10 (or 10′) is easier. The channels 12 (or 12′) may not deform in response to applied pressure. Otherwise, fluid may not flow smoothly. And in turn, temperature at top surface 11 (or 11′) of the pad 10 (or 10′) may not maintain at its predetermined value.

While the invention herein disclosed has been described by means of specific embodiments, numerous modifications and variations could be made thereto by those skilled in the art without departing from the scope and spirit of the invention set forth in the claims.

What is claimed is:

1. A structure in fluid communication with a cooling device or a heating device, comprising:
a pad;
a plurality of elongate, curve channels formed on a top surface of the pad, the channels being connected together wherein one end of the channel is formed as an inlet, the other end of the channel is formed as an outlet, the inlet and the outlet are provided at the same side of the pad, the channel has an arc-shaped periphery, a flat bottom formed on the top surface of the pad, and a lengthwise rib is formed along an inner surface of the channel facing the flat bottom; and
a covering member provided on the top surface of the pad with the channels formed thereon.

2. The structure of claim 1, further comprising:
an inlet coupling provided at the inlet of the channel wherein an outer diameter of one end of the inlet coupling is smaller than an inner diameter of the inlet, a second flat bottom and an upper groove are formed at a periphery of one end of the inlet coupling, the second flat bottom and the upper groove are dimensioned and shaped to be adapted to insert into the flat bottom and the rib respectively for connecting the inlet coupling and the channel together, and the other end of the inlet coupling is formed as a hollow, cylindrical section for permitting cooling or heating fluid to flow therethrough; and
an outlet coupling provided at the outlet of the channel wherein an outer diameter of one end of the outlet coupling is smaller than an inner diameter of the outlet, a third flat bottom and a second upper groove are formed at a periphery of one end of the outlet coupling, the third flat bottom and the second upper groove are dimensioned and shaped to be adapted to insert into the flat bottom and the rib respectively for connecting the outlet coupling and the channel together, and the other end of the outlet coupling is formed as a second hollow, cylindrical section for permitting cooling or heating fluid to flow therethrough.

3. The structure of claim 1, wherein the covering member is a sponge.

4. The structure of claim 1, further comprising a pattern printed on a bottom surface of the pad opposite the channels.
